(19) United States
(12) Patent Application Publication
(10) Pub. No.: US 2021/0230083 A1
(43) Pub. Date: Jul. 29, 2021

(54) Dealkylation and Transalkylation of Heavy Aromatic Hydrocarbons

(71) Applicant: ExxonMobil Chemical Patents Inc., Baytown, TX (US)

(72) Inventors: Todd E. Detjen, Houston, TX (US); Jeevan S. Abichandani, Houston, TX (US)

(21) Appl. No.: 16/332,385

(22) PCT Filed: Feb. 10, 2017
(86) PCT No.: PCT/US2017/017307

§ 371 Date: Mar. 12, 2019

Related U.S. Application Data
(60) Provisional application No. 62/403,754, filed on Oct. 4, 2016.

(51) Int. Cl. C07C 4/18; C07C 5/27; C07C 6/12; C07C 7/20

(52) U.S. Cl. CPC C07C 4/18; C07C 5/2737; C07C 15/04; C07C 7/20; C07C 6/126

(57) ABSTRACT

A process for producing xylene from C₉₊ aromatic hydrocarbons comprises contacting a first feedstock comprising C₉₊ aromatic hydrocarbons with a first catalyst in the presence of hydrogen under effective vapor phase dealkylation conditions to dealkylate part of the C₉₊ aromatic hydrocarbons and produce a first product comprising benzene and unreacted C₉₊ aromatic hydrocarbons. A second feedstock comprising toluene is contacted with a second catalyst in the presence of hydrogen under effective vapor phase toluene disproportionation conditions to disproportionate at least part of the toluene and produce a second product comprising para-xylene. A third feedstock comprising C₉₊ aromatic hydrocarbons and benzene and/or toluene is contacted with a third catalyst in the presence of hydrogen under effective liquid phase C₉₊ transalkylation conditions to transalkylate at least part of the C₉₊ aromatic hydrocarbons and produce a third product comprising xylenes.

### PRIORITY

This application claims priority to and the benefit of U.S. Provisional Application Ser. No. 62/403,754, filed Oct. 4, 2016, the disclosure of which is incorporated herein by reference in its entirety.

### TECHNICAL FIELD

This disclosure relates to transalkylation of heavy (C₉₊) aromatic hydrocarbon feedstocks to produce xylenes, particularly para-xylene.

### BACKGROUND

Xylenes are important aromatic hydrocarbons, for which the worldwide demand is steadily increasing. The demand for xylenes, particularly para-xylene, has increased in proportion to the increase in demand for polyester fibers and film and typically grows at a rate of 5-7% per year. An important source of xylenes and other aromatic hydrocarbons is catalytic reformate, which is produced by contacting a mixture of petroleum naphtha and hydrogen with a strong hydrogenation/dehydrogenation catalyst, such as platinum, on a moderately acidic support, such as a halogen-treated alumina. The resulting reformate is a complex mixture of paraffins and the C₆ to C₈ aromatics, in addition to a significant quantity of heavier aromatic hydrocarbons. After removing the light (C₅₋) paraffinic components, the remainder of the reformate is normally separated into C₇₋, C₈ and C₉₊-containing fractions using a plurality of distillation steps. The C₈-containing fraction is then fed to a xylene production loop where para-xylene is recovered, generally by adsorption or crystallization, and the resultant para-xylene depleted stream is subjected to catalytic conversion to isomerize the xylenes back towards equilibrium distribution and to reduce the level of ethylbenzene that would otherwise build up in the xylene production loop.

However, the quantity of xylene available from reformate C₈ fractions is limited. As a result, refineries have also focused on the production of xylene by transalkylation of heavy (C₉₊) aromatic hydrocarbons (both from reformate and other sources) with benzene and/or toluene over noble metal-containing zeolite catalysts. For example, U.S. Pat. No. 5,942,651 discloses a process for the transalkylation of heavy aromatics comprising contacting a feed comprising C₉₊ aromatic hydrocarbons and toluene with a first catalyst composition comprising a molecular sieve having a constraint index ranging from 0.5 to 3, such as ZSM-12, and a hydrogenation component under transalkylation reaction conditions to produce a transalkylation reaction product comprising benzene and xylene. The transalkylation reaction product is then contacted with a second catalyst composition which comprises a molecular sieve having a constraint index ranging from 3 to 12, such as ZSM-5, and which may be in a separate bed or a separate reactor from the first catalyst composition, under conditions to remove benzene co-boilers in the product.

One problem associated with heavy aromatics transalkylation processes is catalyst aging since, as the catalyst loses activity with increasing time on stream, higher temperatures tend to be required to maintain constant conversion. When the maximum reactor temperature is reached, the catalyst needs to be replaced or regenerated, normally by oxidation. In particular, it has been found that the aging rate of existing transalkylation catalysts is strongly dependent on the presence in the feed of aromatic compounds having alkyl substituents with two or more carbon atoms, such as ethyl and propyl groups. Thus these compounds tend to undergo reactions such as disproportionation and dealkylation/realkylation to produce C₁₀₊ coke precursors.

To address the problem of C₉₊ feeds containing high levels of ethyl and propyl substituents, US Publication No. 2009/0112034 discloses a catalyst system adapted for transalkylation of a C₉₊ aromatic feedstock with a C₆-C₇ aromatic feedstock comprising: (a) a first catalyst comprising a first molecular sieve having a Constraint Index in the range of 3-12 and 0.01 to 5 wt. % of at least one source of a first metal element of Groups 6-10; and (b) a second catalyst comprising a second molecular sieve having a Constraint Index less than 3 and 0 to 5 wt. % of at least one source of a second metal element of Groups 6-10, wherein the weight ratio of said first catalyst to said second catalyst is in the range of 5:95 to 75:25. The first catalyst, which is optimized for dealkylation of the ethyl and propyl groups in the feed, is located in front of the second catalyst, which is optimized for transalkylation, when they are brought into contact with a C₉₊ aromatic feedstock and a C₆-C₇ aromatic feedstock in the presence of hydrogen.

However, despite these and other advances, there remains a number of unresolved problems with existing C₉₊ aromatic conversion processes. One such problem is that the xylenes produced by the transalkylation step are in equilibrium concentration, in which the para-isomer generally comprises only about 22% of the total isomer mixture. Thus, maximizing para-xylene production requires circulation of large volumes of C₈ aromatics in the xylene production loop. In addition, xylene isomerization processes are generally limited by equilibrium constraints as to the per pass conversion of meta- and ortho-xylene to the desired para-isomer, again leading to process inefficiencies.

### BRIEF SUMMARY

According to the present disclosure, it has now been found that the para-xylene yield and production efficiency in C₉₊ aromatic hydrocarbon conversion processes can be improved by supplying the product of an initial dealkylation step, together with fresh and/or recycled toluene, to a disproportionation reaction zone. By suitable selection of the disproportionation catalyst, the toluene can be selectively converted to para-xylene in the disproportionation reaction zone without significant conversion of the C₉₊ aromatic hydrocarbon component. After separation of the para-rich xylene fraction, the remaining C₉₊ aromatic hydrocarbon component can be transalkylated with benzene and/or toluene, preferably benzene produced by the toluene disproportionation reaction, to produce additional xylenes. In addition, carrying the transalkylation in the liquid phase allows for an improved lifetime of the catalysts used and/or an improved selectivity for production of aromatics having the desired number of carbons (such as C₈ aromatics) at lower severity reaction conditions while minimizing energy consumption.

Thus, in one aspect, the present disclosure relates to a process for producing xylene from C₉₊ aromatic hydrocarbons, the process comprising:

(a) contacting a first feedstock comprising C₉₊ aromatic hydrocarbons with a first catalyst in the presence of hydrogen under effective vapor phase dealkylation conditions to dealkylate part of the C₉₊ aromatic hydrocarbons and produce a first product comprising benzene and unreacted C₉₊ aromatic hydrocarbons;
(b) contacting a second feedstock comprising toluene with a second catalyst in the presence of hydrogen under effective vapor phase toluene disproportionation conditions to disproportionate at least part of the toluene and produce a second product comprising para-xylene; and
(c) contacting a third feedstock comprising C₉₊ aromatic hydrocarbons and benzene and/or toluene with a third catalyst in the presence of 0 wt. % or more of hydrogen under effective liquid phase C₉₊ transalkylation conditions to transalkylate at least part of the C₉₊ aromatic hydrocarbons and produce a third product comprising xylenes.

In a further aspect, the present disclosure relates to a process for producing xylene from C₉₊ aromatic hydrocarbons, the process comprising:

(a) contacting a first feedstock comprising C₉₊ aromatic hydrocarbons with a first catalyst in the presence of hydrogen under effective vapor phase dealkylation conditions to dealkylate part of the C₉₊ aromatic hydrocarbons and produce a first product comprising benzene and C₉₊ aromatic hydrocarbons;
(b) contacting at least part of the first product and toluene with a second catalyst in the presence of hydrogen under effective vapor phase toluene disproportionation conditions to disproportionate at least part of the toluene and produce a second product comprising para-xylene, benzene, toluene and C₉₊ aromatic hydrocarbons;
(c) contacting at least part of the C₉₊ aromatic hydrocarbons from the second product and benzene and/or toluene with a third catalyst in the presence of hydrogen under effective liquid phase C₉₊ transalkylation conditions to transalkylate at least part of the C₉₊ aromatic hydrocarbons and produce a third product comprising xylenes; and
(d) separating para-xylene from at least the second product.

### BRIEF DESCRIPTION OF THE DRAWINGS

FIG. 1 shows examples of the mole fraction of a feed in the liquid phase at various temperature and pressure conditions.

FIG. 2 is a flow diagram of a C₉ aromatic hydrocarbon transalkylation process according to one embodiment of the present disclosure.

### DETAILED DESCRIPTION OF THE EMBODIMENTS

### Definitions and Overview

As used herein, the numbering scheme for the Periodic Table Groups is as described in Chemical and Engineering News, 63(5), 27 (1985).

As used in this specification, the term “framework type” is used in the sense described in the “Atlas of Zeolite Framework Types,” 2001.

The term “aromatic” is used herein in accordance with its art-recognized scope which includes alkyl substituted and unsubstituted mono and polynuclear compounds.

The term “catalyst” is used interchangeably with the term “catalyst composition”.

The term “ethyl-aromatic compounds” means aromatic compounds having an ethyl group attached to the aromatic ring. The term “propyl-aromatic compounds” means aromatic compounds having a propyl group attached to the aromatic ring.

The term “Cₙ” hydrocarbon wherein n is an positive integer, e.g., 1, 2, 3, 4, 5, 6, 7, 8, 9, 10, 11, 12, as used herein means a hydrocarbon having n number of carbon atom(s) per molecular. For example, Cₙ aromatics means an aromatic hydrocarbon having n number of carbon atom(s) per molecule. The term “Cₙ₊” hydrocarbon wherein n is an positive integer, e.g., 1, 2, 3, 4, 5, 6, 7, 8, 9, 10, 11, 12, as used herein means a hydrocarbon having at least n number of carbon atom(s) per molecule. The term “Cₙ₊” hydrocarbon wherein n is an positive integer, e.g., 1, 2, 3, 4, 5, 6, 7, 8, 9, 10, 11, 12, as used herein means a hydrocarbon having no more than n number of carbon atom(s) per molecule.

The term “effective vapor phase dealkylation (or toluene disproportionation) conditions” means that the relevant reaction is conducted under conditions of temperature and pressure such that at least part of the aromatic components of the reaction mixture are in the vapor phase. In some embodiments, the mole fraction of the aromatic components in the vapor phase, relative to the total aromatics in the reaction mixture, can be at least 0.75, such as at least 0.85 or 0.95, up to 1 (all the aromatic components in the vapor phase).

The term “effective liquid phase C₉₊ transalkylation conditions” means that the transalkylation reaction is conducted under conditions of temperature and pressure such that at least part of the aromatic components of the transalkylation reaction mixture are in the liquid phase. In some embodiments, the mole fraction of aromatic compounds in the liquid phase, relative to the total aromatics, can be at least 0.01, or at least 0.05, or at least 0.08, or at least 0.1, or at least 0.15, or at least 0.2, or at least 0.3, or at least 0.4, or at least 0.5, and optionally up to having substantially all aromatic compounds in the liquid phase.

The term “mordenite” as used herein includes, but is not limited to, a mordenite zeolite having a very small crystal size and having a high mesopore surface area made by the particular selection of the synthesis mixture composition, as disclosed in WO 2016/126431.

The term “xylenes” as used herein is intended to include a mixture of the isomers of xylene of ortho-xylene, meta-xylene and para-xylene.

Described herein are various processes for producing xylene from C₉₊ aromatic hydrocarbons. In these processes, a first feedstock comprising C₉₊ aromatic hydrocarbons is contacted with a first catalyst in the presence of hydrogen under effective vapor phase dealkylation conditions to dealkylate part of the C₉₊ aromatic hydrocarbons and produce a first product comprising benzene and unreacted C₉₊ aromatic hydrocarbons. A second feedstock comprising toluene, normally together with at least part of the first product, is then contacted with a second catalyst in the presence of hydrogen under effective vapor phase toluene disproportionation conditions to disproportionate at least part of the toluene and produce a second product comprising para-xylene. A third feedstock comprising C₉₊ aromatic hydrocarbons, such as from the second product, together with benzene and/or toluene is then contacted with a third catalyst in the presence of hydrogen under effective liquid phase C₉₊ transalkylation conditions to transalkylate at least part of the C₉₊ aromatic hydrocarbons and produce a third product comprising xylenes. Para-xylene can be recovered from the second product and the third product.

Each of the first, second and third catalysts can be housed in a separate reactor or, where desired, two or more of the catalysts can be accommodated in the same reactor. For example, the first and second catalysts beds can be arranged in separate catalyst beds stacked one on top of the other in a single reactor.

### C₉₊ Aromatic Hydrocarbon Feedstock

The aromatic feed used in the present process comprises one or more aromatic hydrocarbons containing at least 9 carbon atoms. Specific C₉₊ aromatic compounds found in a typical feed include mesitylene (1,3,5-trimethylbenzene), durene (1,2,4,5-tetramethylbenzene), hemimellitene (1,2,4-trimethylbenzene), pseudocumene (1,2,4-trimethylbenzene), 1,2-methylethylbenzene, 1,3-methylethylbenzene, 1,4-methylethylbenzene, propyl-substituted benzenes, butyl-substituted benzenes, and dimethylethylbenzenes. Suitable sources of the C₉₊ aromatics are any C₉₊ fraction from any refinery process that is rich in aromatics. This aromatics fraction may contain a substantial proportion of C₉₊ aromatics, e.g., at least 50 wt. %, such as at least 80 wt. % C₉₊ aromatics, wherein preferably at least 80 wt. %, and more preferably more than 90 wt. %, of the hydrocarbons will range from C₉ to C₁₂. Typical refinery fractions which may be useful include catalytic reformate, FCC naphtha or TCC naphtha.

### Dealkylation Stage

The first stage of the present process comprises contacting the C₉₊ aromatic hydrocarbon feedstock in a first reaction zone with a first catalyst effective to dealkylate C₂₊ alkyl-containing compounds, particularly ethyl-aromatic compounds and propyl-aromatic compounds, to produce mainly benzene and toluene and the corresponding alkenes. The total feed to the first reaction zone therefore normally includes hydrogen (e.g., 0 wt. % or more) to convert the alkenes to the corresponding alkanes In some embodiments, the hydrogen/hydrocarbon molar ratio in the total feed to the first reaction zone may be from 0.05 to 10, for example from 0.1 to 5.

Any known dealkylation catalyst can be used in the first reaction zone. However, in embodiments, the first catalyst comprises a first molecular sieve having a Constraint Index in the range of about 3 to about 12, optionally together with at least one hydrogenation component. In this respect, Constraint Index is a convenient measure of the extent to which an aluminosilicate or other molecular sieve provides controlled access to molecules of varying sizes to its internal structure. For example, molecular sieves which provide a highly restricted access to and egress from its internal structure have a high value for the Constraint Index. Molecular sieves of this kind usually have pores of small diameter, e.g., less than 5 Angstroms. On the other hand, molecular sieves which provide relatively free access to their internal pore structure have a low value for the constraint index, and usually pores of large size. The method by which constraint index is determined is described fully in U.S. Pat. No. 4,016,218, which is incorporated herein by reference for the details of the method.

Suitable molecular sieves for use in the first catalyst composition comprise at least one of ZSM-5, ZSM-11, ZSM-22, ZSM-23, ZSM-35, ZSM-48, ZSM-57 and ZSM-58. ZSM-5 is described in detail in U.S. Pat. No. 3,702,886 and Re. 29,948. ZSM-11 is described in detail in U.S. Pat. No. 3,709,979. ZSM-22 is described in U.S. Pat. Nos. 4,556,477 and 5,336,478. ZSM-23 is described in U.S. Pat. No. 4,076,842. ZSM-35 is described in U.S. Pat. No. 4,016,245. ZSM-48 is more particularly described in U.S. Pat. Nos. 4,234,231 and 4,375,573. ZSM-57 is described in U.S. Pat. No. 4,873,067. ZSM-58 is described in U.S. Pat. No. 4,698,217.

In one preferred embodiment, the first molecular sieve comprises ZSM-5 and especially ZSM-5 having an average crystal size of less than 0.1 micron, for example such that the ZSM-5 crystals have an external surface area in excess of 100 m²/g as determined by the t-plot method for nitrogen physisorption. Suitable ZSM-5 compositions are disclosed in US Publication No. 2015/0298981, the entire contents of which are incorporated herein by reference.

Conveniently, the first molecular sieve has an alpha value in the range of about 100 to about 1500, such as about 150 to about 1000, for example about 150 to about 600. Alpha value is a measure of the cracking activity of a catalyst and is described in U.S. Pat. No. 3,354,078 and in the Journal of Catalysis, Vol. 4, p. 527 (1965); Vol. 6, p. 278 (1966); and Vol. 61, p. 395 (1980), each incorporated herein by reference as to that description. The experimental conditions of the test used herein include a constant temperature of 538° C. and a variable flow rate as described in detail in the Journal of Catalysis, Vol. 61, page 395.

Generally, the first molecular sieve is an aluminosilicate having a silica to alumina molar ratio of less than 1000, typically from about 10 to about 100.

Typically, the first catalyst composition comprises at least 1 wt. %, preferably at least 10 wt. %, more preferably at least 25 wt. %, and most preferably at least 50 wt. %, of the first molecular sieve. In one embodiment, the first catalyst composition comprises from 55 to 80 wt. % of the first molecular sieve.

Additionally or alternatively, the first catalyst composition may comprise a combination of the first molecular sieve having a Constraint Index in the range of about 3 to about 12 and an additional molecular sieve having a Constraint Index less than 3, such as zeolite beta, mordenite or faujasite. For example, the first catalyst composition may comprise a combination of ZSM-5 and mordenite.

In addition to a molecular sieve having a Constraint Index in the range of about 3 to about 12, the first catalyst composition comprises at least one hydrogenation component, such as at least one metal or compound thereof of Groups 6 to 12 of the Periodic Table of the Elements. Suitable hydrogenation components include platinum, palladium, iridium, rhenium and mixtures and compounds thereof, preferably platinum, rhenium and compounds thereof. In some embodiments, the first catalyst composition comprises two or more hydrogenation components including a first metal or compound thereof selected from platinum, palladium, iridium, rhenium and mixtures thereof and a second metal or compound chosen so as to lower the benzene saturation activity of the first metal. Examples of suitable second metals include at least one of copper, silver, gold, ruthenium, iron, tungsten, molybdenum, cobalt, nickel, tin and zinc. Conveniently, the first metal is present in the first catalyst in an amount from 0.001 to 1 wt %, such as from 0.01 to 0.1 wt %, of the first catalyst and the second metal is present in the first catalyst in amount from 0.001 to 10 wt %, 0.1 to 1 wt %, of the first catalyst.

In some embodiments, the first metal comprises platinum and/or rhenium and the second metal comprises copper and/or tin. In one preferred embodiment, the first metal comprises platinum and the second metal comprises tin, desirably at a molar ratio of platinum to tin from 0.1:1 to 1:1, such as from 0.2:1 to 0.4:1.

In some embodiments, the first catalyst composition may comprise one or more of the hydrogenation components described above on a refractory oxide, with or without the presence of a molecular sieve. Suitable refractory oxides comprise silica, alumina, silica alumina and titania.

The or each hydrogenation component can be incorporated into the first catalyst composition by any known method, including co-crystallization, ion exchange into the composition to the extent a Group 13 element, e.g., aluminum, is in the molecular sieve structure, impregnated therein, or mixed with the molecular sieve and binder. In some embodiments, ion exchange may be preferred. After incorporation of the hydrogenation component(s), the catalyst composition is usually dried by heating at a temperature of 65° C. to 160° C., typically 110° C. to 143° C., for at least 1 minute and generally not longer than 24 hours, at pressures ranging from 100 to 200 kPa-a. Thereafter, the catalyst composition may be calcined in a stream of dry gas, such as air or nitrogen, at temperatures of from 260° C. to 650° C. for 1 to 20 hours. Calcination is typically conducted at pressures ranging from 100 to 300 kPa-a.

The first catalyst composition may be self-bound (that is without a separate binder) or may also comprise a binder or matrix material that is resistant to the temperatures and other conditions employed in the present process. Where such a binder or matrix material is present, it is substantially free of amorphous alumina, since it is found that the exclusion of a binder containing amorphous alumina reduces external catalytic sites for coke production and hence increases catalyst cycle length. One preferred binder material for the first catalyst composition comprises silica since extrusion with silica ensures that the catalyst has high mesoporosity and hence high activity. Alternatively, the binder or matrix material may be a crystalline molecular sieve material, which may be isostructural with, or have a different structure than, the first molecular sieve.

Where the first catalyst composition contains a binder or matrix material, the latter may be present in an amount ranging from 5 to 95 wt. %, and typically from 10 to 60 wt. %, of the total catalyst composition.

Examples of specific catalyst compositions useful in the dealkylation stage of the present process include Pt supported on ZSM-5, Pt-supported on silica or alumina, Pt/Sn on a combination of mordenite and ZSM-5, Re on a combination of mordenite and ZSM-5, and Mo on a combination of mordenite and ZSM-5.

The first catalyst composition may be extruded into particles of any desired shape before being loaded into the first catalyst bed. In some embodiments, it may be desirable to control the shape and size of the catalyst particles so as to maximize the external surface area of the catalyst. For example, it may be desirable to control the catalyst particle configuration such that the particles have a surface to volume ratio of about 80 to <200 inch⁻¹, preferably about 100 to 150 inch⁻¹. Suitable particle configurations for achieving such a surface to volume ratio include grooved cylindrical extrudates and hollow or solid polylobal extrudates, such as quadrulobal extrudates.

In operation, the first reaction zone is maintained under vapor phase conditions effective to dealkylate aromatic hydrocarbons containing C₂₊ alkyl groups in the heavy aromatic feedstock and to saturate the resulting C₂₊ olefins. Suitable conditions for operation of the first catalyst bed comprise a temperature in the range of about 100 to about 800° C., preferably about 300 to about 500° C., a pressure in the range of about 790 to about 7000 kPa-a, preferably about 2170 to 3000 kPa-a, a H₂:HC molar ratio in the range of about 0.01 to about 20, preferably about 1 to about 10, and a WHSV in the range of about 0.01 to about 100 hr⁻¹, preferably about 2 to about 20 hr⁻¹.

The product of the dealkylation stage mainly comprises unreacted C₉₊ aromatic hydrocarbons together with smaller amounts of benzene, toluene, xylenes, and lower alkanes. Some or all, preferably all, of the dealkylation product is then supplied, in some embodiments without an intermediate separation step, to a second reaction zone, which also receives a supply of fresh and/or recycled toluene.

### Toluene Disproportionation Stage

In the second, toluene disproportionation stage of the present process, toluene and at least part of the dealkylation effluent are contacted in the second reaction zone with a second catalyst composition comprising a second molecular sieve and optionally one or more hydrogenation components.

Examples of crystalline molecular sieves useful in the second catalyst composition include intermediate pore size zeolites, such as of ZSM-5, ZSM-11, ZSM-22, ZSM-23, ZSM-35, ZSM-48, ZSM-57, and ZSM-58. Also useful are silicoaluminophosphates (SAPO's), particularly SAPO-5 and SAPO-11 (U.S. Pat. No. 4,440,871) and aluminophosphates (ALPO₄'s), particularly ALPO₄-5, and ALPO₄-11 (U.S. Pat. No. 4,310,440). The entire contents of the above references are incorporated by reference herein. Preferred intermediate pore zeolites include ZSM-5, ZSM-11, ZSM-12, ZSM-35, and MCM-22. Most preferred is ZSM-5, preferably having a silica to alumina molar ratio of at least about 5, preferably at least about 10, more preferably at least 20.

Intermediate pore size molecular sieves useful in the toluene disproportionation stage are particularly those which have been modified to decrease their ortho-xylene sorption rate since these are found to be more selective for the production of para-xylene over the other xylene isomers. The desired decrease in ortho-xylene sorption rate can be achieved by subjecting the molecular sieve, in bound or unbound form, to silicon selectivation through ex situ methods of impregnation or multiple impregnation or in situ methods of trim selectivation; or coke selectivation; or combination of these. Multiple impregnation methods are described, e.g., in U.S. Pat. Nos. 5,365,004; 5,367,099; 5,382,737; 5,403,800; 5,406,015; 5,476,823; 5,495,059; and 5,633,417. Other ex situ selectivations are described in U.S. Pat. Nos. 5,574,199 and 5,675,047. Trim selectivation is described, e.g., in U.S. Pat. Nos. 5,321,183; 5,349,113; 5,475,179; 5,498,814; and 5,607,888. Other silicon selectivations are described, e.g., in U.S. Pat. Nos. 5,243,117; 5,349,114; 5,365,003; 5,371,312; 5,455,213; 5,516,736; 5,541,146; 5,552,357; 5,567,666; 5,571,768; 5,602,066; 5,610,112; 5,612,270; 5,625,104; and 5,659,098. Coke selectivation is described in U.S. Pat. Nos. 5,234,875; 4,581,215; 4,508,836; 4,358,395; 4,117,026; and 4,097,543. All of these patents describing selectivation are incorporated by reference herein.

In particular, it has been found that the second catalyst composition should possess an equilibrium sorption capacity of xylene, which can be either para, meta, ortho or a mixture thereof, frequently para-xylene, since this isomer reaches equilibrium within the shortest time, of at least 1 gram per 100 grams of zeolite measured at 120° C. and a xylene pressure of 4.50.8 mm of mercury and an ortho-xylene sorption time for 30 percent of the xylene sorption capacity of greater than 50, preferably greater than 200, more preferably greater than 1200 (at the same conditions of temperature and pressure). The sorption measurements may be carried out gravimetrically in a thermal balance. The sorption test is described in U.S. Pat. Nos. 4,117,025; 4,159,282; 5,173,461; and Re. 31,782; each of which is incorporated by reference herein.

The second catalyst composition may include the second molecular sieve in bound or unbound form. Where a binder is used, a silica binder may be preferred. Procedures for preparing silica bound ZSM-5 are described in, for example, U.S. Pat. Nos. 4,582,815; 5,053,374; and 5,182,242, incorporated by reference herein. In some embodiments, a zeolite bound zeolite, as described in U.S. Pat. No. 5,665,325, may be employed in the second catalyst composition.

In addition to the molecular sieve described above, the second catalyst composition may include at least one hydrogenation component, such as at least one metal or compound thereof from Groups 4 to 13 of the Periodic Table of the Elements. Suitable metals include platinum, palladium, silver, tin, gold, copper, zinc, nickel, gallium, cobalt, molybdenum, rhodium, ruthenium, manganese, rhenium, tungsten, chromium, iridium, osmium, iron, cadmium, and mixtures (combinations) thereof. Preferred metals include Pd, Pt, Ni, Re, Sn, and a combination of two or more thereof. The metal may be added by cation exchange or by impregnation by known methods in amounts of from about 0.01% to about 10%, such as from about 0.01% to about 5%, by weight of the catalyst.

Examples of specific catalyst compositions useful in the toluene disproportionation stage of the present process include coke selectivated ZSM-5, coke selectivated ZSM-5 with a silicalite binder, silicone selectivated ZSM-5, and rhenium supported on silicone selectivated ZSM-5.

Suitable conditions in the second reaction zone effective for accomplishing high para-xylene selectivity and acceptable toluene conversion levels include a reactor inlet temperature of from about 200° C. to about 550° C., preferably from about 300° C. to about 500° C.; a pressure from about atmospheric to about 5000 psig (100 to 34576 kPa-a), preferably about 20 to about 1000 psig (239 to 6996 kPa-a); a WHSV from about 0.1 to about 20, preferably from about 0.5 to about 10; and a H₂/hydrocarbon mole ratio from about 0 to about 20, preferably from about 0 to about 10. In particular, the conditions are generally selected so that at least the toluene is predominantly in the vapor phase. This process may be conducted in either continuous flow, batch or fluid bed operation. In some embodiments, the second reaction zone may be split between two or more separate reactors. In other embodiments, the first and second reaction zones may be housed in a single reactor.

Under the conditions in the second reaction zone, at least part of the toluene undergoes disproportionation into benzene and a para-xylene selective mixture of xylenes. Using the selectivated catalysts described above, the para-xylene content in the mixed xylenes may be of the order of 90%. In contrast, it is expected that the larger C₉₊ aromatic hydrocarbons will not enter the pores of the selectivated catalyst and will pass through the second reaction zone substantially without conversion. As a result, the product exiting the second reaction zone is composed mainly of a para-xylene rich xylene mixture, benzene, lower alkanes and unreacted toluene and unreacted C₉₊ aromatic hydrocarbons. The disproportionation product is therefore sent to a separation system, normally a distillation train, where the product is separated into:
a gaseous mixture of lower alkanes which can be recovered for use as a fuel;
a benzene stream which is fed to a transalkylation reaction zone as described below;
an unreacted toluene stream which is at least partly recycled to the second reaction zone, but can also be fed to the transalkylation reaction zone as described below;
a para-xylene rich C₈ stream which is fed to a para-xylene recovery loop as described below;
an unreacted C₉₊ aromatic hydrocarbon stream which is at least partly fed to the transalkylation reaction zone as described below and which may also be partly recycled the first reaction zone to complete dealkylation; and
an optional C₁₂₊ bottoms stream which may be purged from the system for use as, for example, a fuel.

### Liquid Phase Transalkylation Stage

In the third, liquid phase transalkylation stage of the present process, at least part of the unreacted C₉₊ aromatic hydrocarbon, benzene and optionally part of the toluene recovered from the disproportionation product are contacted in third liquid phase transalkylation reaction zone with a third catalyst composition comprising a third molecular sieve and optionally one or more hydrogenation components.

In one embodiment, a suitable molecular sieve for the third catalyst composition includes a molecular sieve with a framework structure having a 3-dimensional network of 12-member ring pore channels. Examples of framework structures having a 3-dimensional 12-member ring are the framework structures corresponding to faujasite (such as zeolite X or Y, including USY), *BEA (such as zeolite Beta), BEC (polymorph C of Beta), CIT-1 (CON), MCM-68 (MSE), hexagonal faujasite (EMT), ITQ-7 (ISV), ITQ-24 (IWR), and ITQ-27 (IWV), preferably faujasite, hexagonal faujasite, and Beta (including all polymorphs of Beta). It is noted that the materials having a framework structure including a 3-dimensional network of 12-member ring pore channels can correspond to zeolites, silicoaluminophosphates, aluminophosphates, and/or any other convenient combination of framework atoms.

Additionally or alternately, a suitable transalkylation catalyst includes a molecular sieve with a framework structure having a 1-dimensional network of 12-member ring pore channels, where the pore channel has a pore channel size of at least 6.0 Angstroms, or at least 6.3 Angstroms. The pore channel size of a pore channel is defined herein to refer to the maximum size sphere that can diffuse along a channel. Examples of framework structures having a 1-dimensional 12-member ring pore channel can include, but are not limited to, mordenite (MOR), zeolite L (LTL), and ZSM-18 (MEI). It is noted that the materials having a framework structure including a 1-dimensional network of 12-member ring pore channels can correspond to zeolites, silicoaluminophosphates, aluminophosphates, and/or any other convenient combination of framework atoms.

Additionally or alternately, a suitable transalkylation catalyst includes a molecular sieve having the MWW framework structure. Although the MWW framework structure does not have 12-member ring pore channels, the MWW framework structure does include surface sites that have features similar to a 12-member ring opening. Examples of molecular sieves having MWW framework structure include MCM-22, MCM-49, MCM-56, MCM-36, EMM-10, EMM-10-P, EMM-13, PSH-3, SSZ-25, ERB-1, ITQ-1, ITQ-2, UZM-8, UZM-8HS, UZM-37, MIT-1, and interlayer expanded zeolites. It is noted that the materials having an MWW framework structure can correspond to zeolites, silicoaluminophosphates, aluminophosphates, and/or any other convenient combination of framework atoms.

Additionally or alternately, a suitable transalkylation catalyst includes an acidic microporous material that has a largest pore channel corresponding to a 12-member ring or larger, and/or that has a pore channel size of at least 6.0 Angstroms, or at least 6.3 Angstroms and/or that has another active surface having a size of at least 6.0 Angstroms. It is noted that such microporous materials can correspond to zeolites, silicoaluminophosphates, aluminophosphates, and/or materials that are different from molecular sieve type materials.

The molecular sieve can optionally be characterized based on having a composition with a molar ratio YO₂ over X₂O₃ equal to n, wherein X is a trivalent element, such as aluminum, boron, iron, indium and/or gallium, preferably aluminum and/or gallium, and Y is a tetravalent element, such as silicon, tin and/or germanium, preferably silicon. For example, when Y is silicon and X is aluminum, the molar ratio of YO₂ over X₂O₃ is the silica-to-alumina molar ratio. For a MWW framework molecular sieve, n can be less than about 50, e.g., from about 2 to less than about 50, usually from about 10 to less than about 50, more usually from about 15 to about 40. For a molecular sieve having the framework structure of Beta and/or its polymorphs, n can be about 10 to about 60, or about 10 to about 50, or about 10 to about 40, or about 20 to about 60, or about 20 to about 50, or about 20 to about 40, or about 60 to about 250, or about 80 to about 250, or about 80 to about 220, or about 10 to about 400, or about 10 to about 250, or about 60 to about 400, or about 80 to about 400. For a molecular sieve having the framework structure FAU, n can be about 2 to about 400, or about 2 to about 100, or about 2 to about 80, or about 5 to about 400, or about 5 to about 100, or about 5 to about 80, or about 10 to about 400, or about 10 to about 100, or about 10 to about 80. Optionally, the above n values can correspond to n values for a ratio of silica to alumina in the MWW, *BEA, and/or FAU framework molecular sieve. In such optional aspects, the molecular sieve can optionally correspond to an aluminosilicate and/or a zeolite.

Optionally, the catalyst comprises 0.01 wt. % to 5.0 wt. %, or 0.01 wt. % to 2.0 wt. %, or 0.01 wt. % to 1.0 wt. %, or 0.05 wt. % to 5.0 wt. %, or 0.05 wt. % to 2.0 wt. %, or 0.05 wt. % to 1.0 wt. %, or 0.1 wt. % to 5.0 wt. %, or 0.1 to 2.0 wt. %, or 0.1 wt. % to 1.0 wt. %, of a metal element of Groups 5-11 (according to the IUPAC Periodic Table). The metal element may be at least one hydrogenation component, such as one or more metals selected from Group 5-11 and 14 of the Periodic Table of the Elements, or a mixture of such metals, such as a bimetallic (or other multimetallic) hydrogenation component. Optionally, the metal can be selected from Groups 8-10, such as a Group 8-10 noble metal. Specific examples of useful metals are iron, tungsten, vanadium, molybdenum, rhenium, chromium, manganese, ruthenium, osmium, nickel, cobalt, rhodium, iridium, copper, tin, noble metals such as platinum or palladium, and combinations thereof. Specific examples of useful bimetallic combinations (or multimetallic combinations) are those where Pt is one of the metals, such as Pt/Sn, Pt/Pd, Pt/Cu, and Pt/Rh. In some aspects, the hydrogenation component is palladium, platinum, rhodium, copper, tin, or a combination thereof. The amount of the hydrogenation component can be selected according to a balance between hydrogenation activity and catalytic functionality. For a hydrogenation component including two or more metals (such as a bimetallic hydrogenation component), the ratio of a first metal to a second metal can range from 1:1 to about 1:100 or more, preferably 1:1 to 1:10.

Optionally, a suitable transalkylation catalyst can be a molecular sieve that has a constraint index of 1-12, optionally but preferably less than 3. The constraint index can be determined by the method described in U.S. Pat. No. 4,016,218, which is incorporated herein by reference with regard to the details of determining a constraint index.

Additionally or alternately, a transalkylation catalyst (such as a transalkylation catalyst system) can be used that has a reduced or minimized activity for dealkylation. The Alpha value of a catalyst can provide an indication of the activity of a catalyst for dealkylation. In various aspects, the transalkylation catalyst can have an Alpha value of about 100 or less, or about 50 or less, or about 20 or less, or about 10 or less, or about 1 or less. The alpha value test is a measure of the cracking activity of a catalyst and is described in U.S. Pat. No. 3,354,078 and in the Journal of Catalysis, Vol. 4, p. 527 (1965); Vol. 6, p. 278 (1966); and Vol. 61, p. 395 (1980), each incorporated herein by reference as to that description. The experimental conditions of the test used herein include a constant temperature of 538° C. and a variable flow rate as described in detail in the Journal of Catalysis, Vol. 61, p. 395.

In addition to the third molecular sieve, it may be desirable to incorporate in the third catalyst composition another material that is resistant to the temperatures and other conditions employed in the transalkylation process of the disclosure. Such materials include active and inactive materials and synthetic or naturally occurring zeolites, as well as inorganic materials such as clays, silica, hydrotalcites, perovskites, spinels, inverse spinels, mixed metal oxides, and/or metal oxides such as alumina, lanthanum oxide, cerium oxide, zirconium oxide, and titania. The inorganic material may be either naturally occurring, or in the form of gelatinous precipitates or gels including mixtures of silica and metal oxides.

Use of a material in conjunction with each molecular sieve, i.e., combined therewith or present during its synthesis, which itself is catalytically active, may change the conversion and/or selectivity of the catalyst composition. Inactive materials suitably serve as diluents to control the amount of conversion so that transalkylated products can be obtained in an economical and orderly manner without employing other means for controlling the rate of reaction. These catalytically active or inactive materials may be incorporated into, for example, alumina, to improve the crush strength of the catalyst composition under commercial operating conditions. It is desirable to provide a catalyst composition having good crush strength because in commercial use, it is desirable to prevent the catalyst composition from breaking down into powder-like materials.

Naturally occurring clays that can be composited with each molecular sieve as a binder for the catalyst composition include the montmorillonite and kaolin family, which families include the subbentonites, and the kaolins commonly known as Dixie, McNamee, Ga. and Florida clays or others in which the main mineral constituent is halloysite, kaolinite, dickite, nacrite or anauxite. Such clays can be used in the raw state as originally mined or initially subjected to calcination, acid treatment or chemical modification.

In addition to the foregoing materials, each molecular sieve (and/or other microporous material) can be composited with a binder or matrix material, such as an inorganic oxide selected from the group consisting of silica, alumina, zirconia, titania, thoria, beryllia, magnesia, lanthanum oxide, cerium oxide, manganese oxide, yttrium oxide, calcium oxide, hydrotalcites, perovskites, spinels, inverse spinels, and combinations thereof, such as silica-alumina, silica-magnesia, silica-zirconia, silica-thoria, silica-beryllia, silica-titania, as well as ternary compositions such as silica-alumina-thoria, silica-alumina-zirconia, silica-alumina-magnesia and silica-magnesia-zirconia. It may also be advantageous to provide at least a part of the foregoing porous matrix binder material in colloidal form so as to facilitate extrusion of the catalyst composition.

In some aspects, a molecular sieve (and/or other microporous material) can be used without an additional matrix or binder. In other aspects, a molecular sieve/microporous material can be admixed with a binder or matrix material so that the final catalyst composition contains the binder or matrix material in an amount ranging from 5 to 95 wt. %, and typically from 10 to 60 wt. %.

Prior to use, steam treatment of the catalyst composition may be employed to minimize the aromatic hydrogenation activity of the catalyst composition. In the steaming process, the catalyst composition is usually contacted with from 5 to 100% steam, at a temperature of at least 260° C. to 650° C. for at least one hour, specifically 1 to 20 hours, at a pressure of 100 to 2590 kPa-a.

A hydrogenation component can be incorporated into the catalyst composition by any convenient method. Such incorporation methods can include co-crystallization, exchange into the catalyst composition, liquid phase and/or vapor phase impregnation, or mixing with the molecular sieve and binder, and combinations thereof. For example, in the case of platinum, a platinum hydrogenation component can be incorporated into the catalyst by treating the molecular sieve with a solution containing a platinum metal-containing ion. Suitable platinum compounds for impregnating the catalyst with platinum include chloroplatinic acid, platinous chloride and various compounds containing the platinum ammine complex, such as Pt(NH₃)₄Cl₂.H₂O or (NH₃)₄Pt(NO₃)₂.H₂O. Palladium can be impregnated on a catalyst in a similar manner.

Alternatively, a compound of the hydrogenation component may be added to the molecular sieve when it is being composited with a binder, or after the molecular sieve and binder have been formed into particles by extrusion or pelletizing. Still another option can be to use a binder that is a hydrogenation component and/or that includes a hydrogenation component.

After treatment with the hydrogenation component, the catalyst is usually dried by heating at a temperature of 65° C. to 160° C., typically 110° C. to 143° C., for at least 1 minute and generally not longer than 24 hours, at pressures ranging from 100 to 200 kPa-a. Thereafter, the molecular sieve may be calcined in a stream of dry gas, such as air or nitrogen, at temperatures of from 260° C. to 650° C. for 1 to 20 hours. Calcination is typically conducted at pressures ranging from 100 to 300 kPa-a.

In addition, prior to contacting the catalyst composition with the hydrocarbon feed, the hydrogenation component can optionally be sulfided. This is conveniently accomplished by contacting the catalyst with a source of sulfur, such as hydrogen sulfide, at a temperature ranging from about 320° C. to 480° C. The source of sulfur can be contacted with the catalyst via a carrier gas, such as hydrogen or nitrogen. Sulfiding per se is known and sulfiding of the hydrogenation component can be accomplished without more than routine experimentation by one of ordinary skill in the art in possession of the present disclosure.

Generally, the conditions employed in a liquid phase transalkylation process can include a temperature of about 400° C. or less, or about 360° C. or less, or about 320° C. or less, and/or at least about 100° C., or at least about 200° C., such as between 100° C. to 400° C., or 100° C. to 340° C., or 230° C. to 300° C.; a pressure of 2.0 MPa-g to 10.0 MPa-g, or 3.0 MPa-g to 8.0 MPa-g, or 3.5 MPa-g to 6.0 MPa-g; an H₂: hydrocarbon molar ratio of 0 to 20, or 0.01 to 20, or 0.1 to 10; and a weight hourly space velocity (“WHSV”) for total hydrocarbon feed to the reactor(s) of 0.1 to 100 hr⁻¹, or 1 to 20 hr⁻¹. Optionally, the pressure during transalkylation can be at least 4.0 MPa-g. It is noted that H₂ is not necessarily required during the reaction, so optionally the transalkylation can be performed without introduction of H₂. The feed can be exposed to the transalkylation catalyst under fixed bed conditions, fluidized bed conditions, or other conditions that are suitable when a substantial liquid phase is present in the reaction environment.

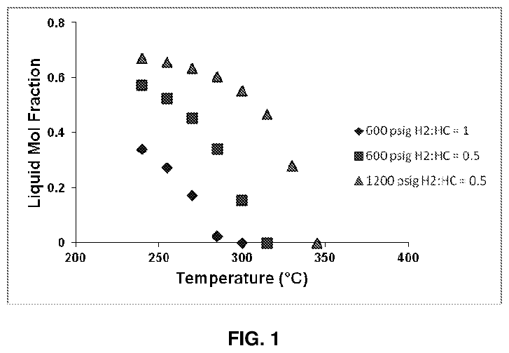

In addition to staying within the general conditions above, the transalkylation conditions can be selected so that a desired amount of the hydrocarbons (reactants and products) in the reactor are in the liquid phase. Referring now to FIG. 1, where it shows calculations for the amount of liquid that should be present for a feed corresponding to a 1:1 mixture of toluene and mesitylene at several conditions that are believed to be representative of potential transalkylation conditions. The calculations in FIG. 1 show the mole fraction that is in the liquid phase as a function of temperature. The three separate groups of calculations shown in FIG. 1 correspond to a vessel containing a specified pressure based on introducing specified relative molar volumes of the toluene/mesitylene feed and H₂ into the reactor. One data set corresponds to a 1:1 molar ratio of toluene/mesitylene feed and H₂ at 600 psig (˜4 MPa-g). A second data set corresponds to a 2:1 molar ratio of toluene/mesitylene feed and H₂ at 600 psig (˜4 MPa-g). A third data set corresponds to a 2:1 molar ratio of toluene/mesitylene feed and H₂ at 1200 psig (˜8 MPa-g).

As shown in FIG. 1, temperatures below about 260° C. can lead to formation of a substantial liquid phase (liquid mole fraction of at least 0.1) under all of the calculated conditions, including the combination of the lower pressure (600 psig) and the lower ratio of feed to hydrogen (1:1) shown in FIG. 1. It is noted that based on a ratio of feed to hydrogen of 1:1, a total pressure of 600 psig corresponds to partial pressure of aromatic feed of about 300 psig. Higher temperatures up to about 320° C. can also have a liquid phase (at least 0.01 mole fraction), depending on the pressure and relative amounts of reactants in the environment. More generally, temperatures such as up to 360° C. or up to 400° C. or greater can also be used for liquid phase transalkylation, so long as the combination of temperature and pressure in the reaction environment can result in a liquid mole fraction of at least 0.01. It is noted that conventional transalkylation conditions typically involve temperatures greater than 350° C. and/or pressures below 4 MPag, but such conventional transalkylation conditions do not include a combination of pressure and temperature that results in a liquid mole fraction of at least 0.01.

The resulting effluent from the liquid phase transalkylation process can have a xylene yield, relative to the total weight of the hydrocarbons in the effluent, of at least about 4 wt. %, or at least about 6 wt. %, or at least about 8 wt. %, or at least about 10 wt. %. Other major components of the transalkylation effluent include benzene, toluene and residual C₉ aromatic hydrocarbons. Separation of these components can be achieved using the same or a different separation system as that used to separate the products of the toluene disproportionation stage. In particular, the xylene can be recovered and supplied to a para-xylene recovery loop, while the benzene and residual C₉₊ aromatic hydrocarbons can be recycled to the liquid phase transalkylation reactor and the toluene can be removed and either recycled to disproportionation or liquid phase transalkylation or both.

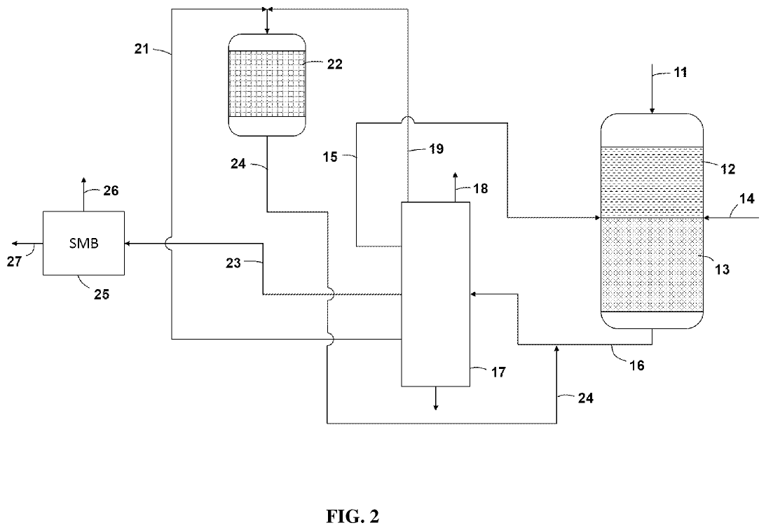

One embodiment of the present process for producing xylenes, and particularly para-xylenes, from C₉₊ aromatic hydrocarbons is shown in FIG. 2, in which a fresh C₉ aromatic hydrocarbon feed is supplied by line 11 to a dealkylation reaction zone 12, which also receives a supply of hydrogen (not shown). The dealkylation reaction zone 12 houses a first catalyst composition comprising a molecular sieve having a Constraint Index of 3 to 12, such as ZSM-5, and a hydrogenation metal, such as platinum. The dealkylation reaction zone 12 is operated under vapor phase dealkylation conditions such that at least some of the aromatic hydrocarbons containing C₂₊ alkyl groups are dealkylated to produce benzene, toluene and xylenes and the corresponding C₂₊ alkenes. The latter are hydrogenated under the conditions in the dealkylation reaction zone 12 so that the major components of the dealkylation effluent are residual C₉₊ aromatic hydrocarbons (typically at least 15 wt. % up to 75 or 80 wt. % of the effluent), benzene, toluene, xylenes and lower alkanes (mostly ethane and propane).

In the embodiment shown in FIG. 2, the dealkylation reaction zone 12 is located in the same reactor as, and upstream from, a disproportionation reaction zone 13 which receives the entire effluent from the dealkylation reaction zone 12 without intermediate separation. In addition, fresh toluene is supplied to the disproportionation reaction zone 13 via line 14 and recycled toluene is supplied via line 15. The disproportionation reaction zone 13 houses a second catalyst composition comprising a molecular sieve having a Constraint Index of 3 to 12, such as ZSM-5, and a hydrogenation metal, such as platinum or rhenium. The reaction zone 13 is maintained under vapor phase conditions effective to disproportionate at least part of the toluene into benzene and a para-selective mixture of xylene isomers, typically containing of the order of 90% para-xylene. It is expected that the larger C₉₊ aromatic hydrocarbons will not enter the pores of the selectivated catalyst and will pass through the reaction zone 13 substantially unconverted. Optionally, the reaction zone 13 may be split into two or more separate reaction zones.

The effluent from the disproportionation reaction zone 13 is collected in line 16 and fed to a fractionation system 17, where the light gases are removed via line 18 and the benzene and residual C₉₊ aromatic hydrocarbons are collected in lines 19 and 21, respectively, and fed to a transalkylation reaction zone 22. In some embodiments (not shown), part of the residual C₉₊ aromatic hydrocarbons collected in line 21 is recycled to the dealkylation reaction zone 12. In addition, the residual toluene is removed via line 15 for recycle to the disproportionation reaction zone 13, while the para-xylene rich C₈ component is separated via line 23 for recovery of the desired para-xylene product.

The transalkylation reaction zone 22 houses a third catalyst composition comprising a molecular sieve, typically having a Constraint Index less than 3, such as MCM-49, and a hydrogenation component, such as platinum or palladium. The reaction zone 22 is maintained under liquid phase conditions effective for transalkylation of at least part of the residual C₉₊ aromatic hydrocarbons supplied by line 21 with at least part of the benzene supplied by line 19 to produce toluene and an equilibrium mixture of xylene isomers. The effluent from the transalkylation reaction zone 22 is collected in line 24 and fed to the fractionation system 17, typically after combination with the effluent from the reaction zone 13 in line 14.

The para-xylene rich C₈ stream collected in line 23 typically has from above equilibrium amounts of para-xylene (above 24 wt. %) up to about 60 wt. %. This para-xylene rich C₈ stream is initially supplied to a para-xylene recovery unit, such as, for example, a para-xylene extraction unit or a simulated moving bed column (SMB) 25, where the para-xylene is selectively adsorbed and, after treatment with a suitable desorbant, such as for example paradiethylbenzene, paradifluorobenzene, diethylbenzene, toluene or mixtures thereof, is recovered via line 26 for further purification. After separation of the para-xylene, the remaining para-xylene depleted steam is fed by line 27 to a xylene isomerization section (not shown) which can be operated in the gas (i.e., vapor) phase or the liquid phase to return the xylenes in para-xylene depleted steam back to equilibrium concentration before the isomerized stream is recycled back to the SMB 25.

While this disclosure has included description and illustration of particular embodiments, those of ordinary skill in the art will appreciate that this disclosure lends itself to variations not necessarily illustrated herein. For this reason, then, reference should be made solely to the appended claims for purposes of determining the scope of the present invention.

What is claimed is:

1. A process for producing xylene from C₉₊ aromatic hydrocarbons, the process comprising:
(a) contacting a first feedstock comprising C₉₊ aromatic hydrocarbons with a first catalyst in the presence of 0 wt. % or more of hydrogen under effective vapor phase dealkylation conditions to dealkylate part of the C₉₊ aromatic hydrocarbons and produce a first product comprising benzene and unreacted C₉₊ aromatic hydrocarbons;
(b) contacting a second feedstock comprising toluene with a second catalyst in the presence of hydrogen under effective vapor phase toluene disproportionation conditions to disproportionate at least part of the toluene and produce a second product comprising para-xylene; and
(c) contacting a third feedstock comprising C9+ aromatic hydrocarbons and benzene and/or toluene with a third catalyst in the presence of hydrogen under effective liquid phase C₉₊ transalkylation conditions to transalkylate at least part of the C₉₊ aromatic hydrocarbons and produce a third product comprising xylenes.

2. The process of claim 1, wherein the second feedstock comprises at least a portion of the unreacted C₉₊ aromatic hydrocarbons of the first product.

3. The process of claim 2, wherein the second product further comprises at least a portion of the unreacted C₉₊ aromatic hydrocarbons of the first product.

4. The process of claim 3, wherein the third feedstock comprises at least a portion of the unreacted C₉₊ aromatic hydrocarbons of the second product.

5. The process of claim 1, wherein at least a portion of the first feedstock is fresh C₉₊ aromatic hydrocarbons and/or at least a portion of the third feedstock is recycled C₉₊ aromatic hydrocarbons.

6. The process of claim 1, further comprising the steps of separating any xylenes from the first product and/or the second product and/or the third product, and then supplying the separated xylenes to a para-xylene recovery unit to recover para-xylene.

7. The process of claim 1, wherein the first product and/or the second product and/or the third product further comprises ortho-xylene and meta-xylenes, and further comprising the steps of separating the ortho-xylene and meta-xylenes, and then isomerizing the separated ortho-xylene and meta-xylenes to form additional para-xylene.

8. The process of claim 1, wherein the effective vapor phase toluene disproportionation conditions comprise a temperature of about 200° C. to about 550° C., a pressure from about atmospheric to about 5000 psig (100 to 34576 kPa-a), or a combination thereof, and a molar ratio of H₂ to hydrocarbons in the second feedstock of about 0 to about 10.

9. The process of claim 1, wherein the effective vapor phase dealkylation conditions comprise a temperature of about 200° C. to about 600° C., a total pressure of about 5 MPa-g or less, or a combination thereof, and a molar ratio of H₂ to hydrocarbons in the first feedstock of about 0 to about 10.

10. The process of claim 1, wherein the effective liquid phase C₉₊ transalkylation conditions are liquid phase and comprise a temperature of about 200° C. to about 500° C., a total pressure of about 10 MPa-g or less, or a combination thereof, and a molar ratio of H₂ to hydrocarbons in the third feedstock of about 0 to about 10.

11. The process of claim 1, wherein the first catalyst comprises ZSM-5.

12. The process of claim 1, wherein the second catalyst comprises silicone selectivated or carbon selectivated ZSM-5.

13. The process of claim 12, wherein the second catalyst further comprises 0.01 wt. % to 5 wt. % of a metal from selected from the group consisting of Cu, Pd, Pt, Ni, Re, Rh, Sn, and a combination of two or more thereof.

14. The process of claim 1, wherein the third catalyst comprises a molecular sieve having at least one of an MWW framework, a *BEA framework, a BEC framework, a FAU framework, a MOR framework, or a mixture of two or more thereof.

15. The process of claim 1, wherein the third catalyst comprises a molecular sieve having an MWW framework type selected from the group consisting of MCM-22, MCM-36, MCM-49, MCM-56, SSZ-25, MIT-1, EMM-10, EMM-10-P, EMM-12, EMM-13, ITQ-1, ITQ-2, ITQ-30, UZM-8, UZM-8HS, UZM-37, and mixtures of two or more thereof.

16. A process for producing xylene from C₉₊ aromatic hydrocarbons, the process comprising:
(a) contacting a feedstock comprising C₉₊ aromatic hydrocarbons with a first catalyst in the presence of 0 wt. % or more of hydrogen under effective vapor phase dealkylation conditions to dealkylate part of the C9+ aromatic hydrocarbons and produce a first product comprising benzene and C9+ aromatic hydrocarbons;
(b) contacting at least part of the first product and toluene with a second catalyst in the presence of hydrogen under effective vapor phase toluene disproportionation conditions to disproportionate at least part of the toluene and produce a second product comprising para-xylene, benzene, toluene and C9+ aromatic hydrocarbons;
(c) contacting at least part of the C9+ aromatic hydrocarbons from the second product and benzene and/or toluene with a third catalyst in the presence of hydrogen under effective C9+ transalkylation conditions to transalkylate at least part of the C9+ aromatic hydrocarbons and produce a third product comprising xylenes; and
(d) separating para-xylene from at least the second product.

17. The process of claim 16, wherein the contacting in (c) is conducted under liquid phase C₉₊ transalkylation conditions.

18. The process of claim 16, wherein the first product is supplied to the contacting (b) without intermediate separation.

19. The process of claim 16, further comprising separating benzene from the second product and recycling at least part of the separated benzene to the contacting (c).

20. The process of claim 16, further comprising separating toluene from the second product and recycling at least part of the separated toluene to the contacting in (b).

21. The process of claim 16, further comprising separating para-xylene from the second product and the third product.

22. The process of claim 16, wherein the first catalyst comprises ZSM-5 and a hydrogenation component.

23. The process of claim 16, wherein the second catalyst comprises selectivated ZSM-5.

24. The process of claim 16, wherein the third catalyst comprises a molecular sieve having at least one of an MWW framework, a *BEA framework, a BEC framework, a FAU framework, a MOR framework, or a mixture of two or more thereof.

25. The process of claim 23, wherein the third catalyst further comprises 0.01 wt. % to 5 wt. % of a metal from Groups 5-11 and 14 selected from the group consisting of Pd, Pt, Ni, Rh, Sn, and a combination of two or more thereof.
